(19) United States
(12) Patent Application Publication
(10) Pub. No.: US 2025/0263351 A1
(43) Pub. Date: Aug. 21, 2025

(54) PROCESS FOR PRODUCING SYNTHETIC HYDROCARBONS FROM CARBON DIOXIDE

(71) Applicants: ATOMIC ENERGY OF CANADA LIMITED/ ÉNERGIE ATOMIQUE DU CANADA LIMITÉE, Chalk River (CA); EXPANDER ENERGY INC., Calgary (CA)

(72) Inventors: Steve KRESNYAK, Calgary (CA); Gord CRAWFORD, Calgary (CA); Sellathurai SUPPIAH, Ontario (CA); Adrian Vega ZUNIGA, Chalk River (CA); Adriana Gaona GOMEZ, Chalk River (CA)

(73) Assignees: EXPANDER ENERGY INC., Calgary, AB (CA); Canadian Nuclear Laboratories Ltd. / Laboratoires Nucléaires Canadiens Ltée, Chalk River, ON (CA); ATOMIC ENERGY OF CANADA LIMITED / ÉNERGIE ATOMIQUE DU CANADA LIMITÉE, Chalk River, ON (CA)

(21) Appl. No.: 18/859,299

(22) PCT Filed: Apr. 25, 2023
(86) PCT No.: PCT/CA2023/050557

§ 371 Date: Oct. 23, 2024

Related U.S. Application Data
(60) Provisional application No. 63/384,000, filed on Nov. 16, 2022.
(60) Provisional application No. 63/383,390, filed on Nov. 11, 2022.
(60) Provisional application No. 63/334,544, filed on Apr. 25, 2022.

(51) Int. Cl. C07C 1/12

(52) U.S. Cl. CPC C07C 1/12

(57) ABSTRACT

Processes for preparing synthetic hydrocarbons are disclosed herein. An aspect of the process comprises: electrolyzing a feed stream comprising steam and CO₂ with a H₂O/CO₂ (S/C) molar ratio greater than about 2.0 in a high-temperature co-electrolyzer apparatus operating at a co-electrolyzer temperature to produce an enhanced hydrogen rich syngas having a first concentration of H₂, removing at least a portion of H₂ from the enhanced hydrogen rich syngas to provide a hydrogen rich syngas having a second concentration of H₂ that is lower than the first concentration of H₂, and producing at least an FT product stream that comprises synthetic hydrocarbons by reacting the hydrogen rich syngas in a Fischer-Tropsch (FT) reactor. Processes for reducing methane and soot content during production of an enhanced hydrogen rich syngas are also disclosed.

### CROSS REFERENCE TO RELATED APPLICATIONS

This application claims the benefit of U.S. Provisional Application No. 63/383,390 filed Nov. 11, 2022 and entitled Process for producing synthetic hydrocarbons from carbon dioxide; U.S. Provisional Application No. 63/384,000 filed Nov. 16, 2022 and entitled Process for producing synthetic hydrocarbons from carbon dioxide; U.S. Provisional Application No. 63/334,544 filed Apr. 25, 2022 and entitled Process for producing synthetic hydrocarbons from biomass, the entirety of these applications being incorporated herein by reference.

### FIELD

The present disclosure relates generally to a process for preparing synthetic hydrocarbons from CO₂ captured from air or industrial processes.

### BACKGROUND

U.S. Pat. No. 9,631,284 discloses a method and system for creating high value liquid fuels such as gasoline, diesel, jet and alcohols using carbon dioxide and water as the starting raw materials. These methods combine a solid oxide electrolytic cell (SOEC) for the efficient and clean conversion of carbon dioxide and water to hydrogen and carbon monoxide, integrated with a gas-to-liquid fuels producing method.

U.S. Pat. No. 11,214,488 discloses a synthesis gas production process from CO₂ and H₂O by co-electrolysis, wherein the CO₂ and CH₄ content of the produced gas is reduced subsequent to co-electrolysis by the produced gas been additionally fed to a catalytic reactor favoring a reverse water-gas shift reaction and/or steam reforming reaction and/or being fed to a coke-filled container.

This background information is provided for the purpose of making known information believed by the applicant to be of possible relevance to the present invention. No admission is necessarily intended, nor should be construed, that any of the preceding information constitutes prior art against the present invention.

### SUMMARY

The teachings described herein may, in one broad aspect, relate to a process for preparing synthetic hydrocarbons from CO₂ captured from the air or industrial processes. In accordance with one broad aspect of the teachings described herein, which may be used alone or in combination with any other aspects described herein, there is provided a process for preparing synthetic hydrocarbons from a carbon feedstock which comprises:
(a) electrolyzing steam and CO₂ in a high temperature co-electrolyzer to produce oxygen, an enhanced hydrogen rich syngas and heat energy;
(b) treating the enhanced hydrogen rich syngas generated in step a) to generate a concentrated hydrogen stream and a hydrogen rich syngas;
(c) reacting the hydrogen rich syngas in a Fischer Tropsch (FT) reactor to produce the synthetic hydrocarbons, water, heat energy and refinery gas.

The process may further include recycling at least a portion of the water produced in step c), for use in step a).

The process may further include recycling at least a portion of the heat energy produced in step c), for generating electric power for use in step a).

The process may further include recycling at least a portion of the refinery gas produced in step c) to generate electric power for use in step a), as feed to a high temperature co-electrolyzer to produce additional enhanced hydrogen rich syngas, or to generate further heat energy, or any combination thereof.

The process may include the production in step a) of an enhanced hydrogen rich syngas having an H₂:CO molar ratio of greater than 2.2:1, or more preferably it may have an H₂:CO molar ratio of greater than about 2.3:1 but less than about 7:1.

The process may include the use in step a) of a feed to the high temperature electrolyzer that has a H₂O/CO₂ (or steam/carbon (S/C)) molar ratio that is greater than 2.0, or more preferably greater than 2.2, or most preferably between 3.0 and 5.0.

The process may include the use in step a) of a high temperature co-electrolysis step carried out at a co-electrolyzer operating temperature of between about 400° C. and about 1000° C., or more preferably at a temperature between about 600° C. and about 800° C.

In accordance with another broad aspect of the teachings described herein, which may be used alone or in combination with any other aspects described herein, there is provided a process for preparing synthetic hydrocarbons, the process including:
(a) electrolyzing a feed stream comprising steam and CO₂ with a H₂O/CO₂ (S/C) molar ratio greater than about 2.0 in a high-temperature co-electrolyzer apparatus operating at a co-electrolyzer temperature to produce an enhanced hydrogen rich syngas having a first concentration of H₂;
(b) removing at least a portion of H₂ from the enhanced hydrogen rich syngas to provide a hydrogen rich syngas having a second concentration of H₂ that is lower than the first concentration of H₂;
(c) producing at least an FT product stream that comprises synthetic hydrocarbons by reacting the hydrogen rich syngas in a Fischer-Tropsch (FT) reactor.

The process may have a S/C molar ratio greater than about 2.2.

The process may have a S/C molar ratio between about 3.0 and about 5.0.

The process may have a co-electrolyzer temperature between about 400° C. and about 1000° C.

The process may have a co-electrolyzer temperature between about 600° C. and about 800° C.

The process may have an enhanced hydrogen rich syngas having an H₂:CO molar ratio of greater than 2.2:1.

The process may have a H₂:CO molar ratio between about 2.3:1 and about 7:1.

The process may further include recovering an oxygen stream from the high temperature co-electrolyzer apparatus in step a).

The process may have a portion of H₂ removed as a concentrated hydrogen stream.

The process may further include recycling at least a portion of the concentrated hydrogen stream into the high temperature co-electrolyzer apparatus.

The process may further include purifying at least a portion of the concentrated hydrogen stream to produce high purity hydrogen.

The process may have at least a portion of the concentrated hydrogen stream purified via a pressure swing adsorption (PSA) unit.

The process may further include recovering an off gas stream produced by the PSA unit.

The process may have at least a portion of H₂ removed in step b) via a separator apparatus comprising one or more of a membrane, a pressure swing adsorption (PSA) unit or an absorption operation.

The process may further include recovering a process water stream, process steam or both, during steps b) or c).

The process may have a process water stream recycled to provide part of the feed stream of step a).

The process may have a process water stream treated by a water treatment process block prior to use in the feed stream.

The process may have a primary source of the steam in the feed stream provided by the process water stream.

The process may have a process steam recycled for use in generating electricity, providing heat for the high temperature co-electrolyzer apparatus, or both.

The process may have a process steam passed through one or more heat exchangers to provide heat for the high temperature co-electrolyzer apparatus.

The process may further include recovering a FT refinery gas stream from the FT reactor during step c).

The process may further include recycling at least a portion of the FT refinery gas to generate electric power for the high temperature co-electrolyzer apparatus, to provide part of the feed stream, or to generate heat energy, or any combination thereof.

The process may further include removing at least a portion of CO₂ from the FT refinery gas stream.

The process may further include removing at least a portion of CO₂ from the hydrogen-rich syngas prior to producing the at least an FT product stream in the FT reactor.

The process may further include removing at least a portion of CO₂ from the off gas stream.

The process may further include recycling the portion of CO₂ to provide part of the feed stream.

The process may have a high temperature co-electrolyzer comprising a solid oxide electrolytic cell (SOEC).

In accordance with another broad aspect of the teachings described herein, which may be used alone or in combination with any other aspects described herein, there is provided a process for reducing methane and soot content during production of an enhanced hydrogen rich syngas, the process including:
electrolyzing a feed stream comprising steam and CO₂ with a H₂O/CO₂ (S/C) molar ratio greater than about 2.0 in a high temperature co-electrolyzer apparatus operating at a co-electrolyzer temperature to produce an enhanced hydrogen rich syngas comprising carbon monoxide (CO) and having a first concentration of H₂;
wherein the enhanced hydrogen rich syngas has an H₂: CO molar ratio of greater than 2.2:1.

The process may have a S/C molar ratio greater than about 2.2.

The process may have a S/C molar ratio between about 3.0 and about 5.0.

The process may have a co-electrolyzer temperature between about 400° C. and about 1000° C.

The process may have a co-electrolyzer temperature between about 600° C. and about 800° C.

The process may further include removing at least a portion of H₂ from the enhanced hydrogen rich syngas to provide a hydrogen rich syngas having a second concentration of H₂ that is lower than the first concentration of H₂.

Other advantages of the present teachings may become apparent to those of skill in the art upon reviewing the present specification.

### BRIEF DESCRIPTION OF THE DRAWINGS

Embodiments of the present disclosure will be described with reference to the accompanying drawings, wherein like reference numerals denote like parts, and in which:

FIG. 1 is a schematic depicting a process flow diagram of a conventional co-electrolysis to liquid process;

FIG. 2 is a schematic depicting a process flow diagram for a co-electrolysis to liquid process in accordance with an embodiment of the present disclosure;

FIG. 3 is a schematic depicting a process flow diagram for a co-electrolysis to liquid process in accordance with an embodiment of the present disclosure;

FIG. 4 is a series of two comparative graphs depicting the outlet H₂:CO ratio of the high temperature co-electrolyzer syngas product stream as a function of temperature and inlet H₂O:CO₂ molar ratio;

FIG. 5 is a series of two graphs depicting the methane and carbon formation molar content respectively of the outlet stream of the high temperature co-electrolyzer syngas product stream as a function of temperature and inlet H₂O:CO₂ molar ratio;

FIG. 6 is a schematic depicting an embodiment of the process; and

FIG. 7 is a schematic depicting another embodiment of the process.

### DETAILED DESCRIPTION

Various apparatuses or processes will be described below to provide examples of embodiments of the present disclosure. No embodiment described below limits any claims and any claims may cover processes or apparatuses that differ from those described below. The disclosed embodiments are not limited to apparatuses or processes having all of the features of any one apparatus or process described below or to features common to multiple or all of the apparatuses described below. It is possible that an apparatus or process described below is not an embodiment of any claims. Any disclosure of an apparatus or process described below that is not claimed in this document may be the subject matter of another protective instrument, for example, a continuing patent application, and the applicants, inventors or owners do not intend to abandon, disclaim, or dedicate to the public any such embodiments by its disclosure in this document.

Unless defined otherwise, all technical and scientific terms used herein have the same meaning as commonly understood by one of ordinary skill in the art to which this disclosure belongs.

As used herein, the term “syngas” is an abbreviation for “synthesis gas”, which is a mixture comprising hydrogen and carbon monoxide.

As used herein, the term “hydrogen rich syngas” refers to syngas having a H₂:CO molar ratio of about 2:1, such as a range of 1.8:1 to 2.2:1 or any values or subranges therebetween, which may be the desired optimum ratio for use in a given Fischer-Tropsch reaction. For example, molar ratios of 1.81:1, 1.82:1, 1.83:1, 1.84:1, 1.85:1, 1.86:1, 1.87:1, 1.88:1, 1.89:1, 1.90:1, 1.91:1, 1.92:1, 1.93:1, 1.94:1, 1.95:1, 1.96:1, 1.97:1, 1.98:1, 1.99:1, 2.00:1, 2.01:1, 2.02:1, 2.03:1, 2.04:1, 2.05:1, 2.06:1, 2.07:1, 2.08:1, 2.09:1, 2.10:1, 2.11:1, 2.12:1, 2.13:1, 2.14:1, 2.15:1, 2.16:1, 2.17:1, 2.18:1, 2.19:1, and others are considered.

As used herein, the term “enhanced hydrogen rich syngas” refers to syngas having H₂:CO molar ratio of greater than 2.2:1, such as a range of 2.3:1 to 7:1 or any values or subranges therebetween, which contains excess hydrogen which may be separated for other internal or external uses. For example, molar ratios of 2.21:1, 2.22:1, 2.23:1, 2.24:1, 2.25:1, 2.26:1, 2.27:1, 2.28:1, 2.29:1, 2.30:1, 2.35:1, 2.40:1, 2.45:1, 2.50:1, 2.55:1, 2.60:1, 2.65:1, 2.70:1, 2.75:1, 2.80:1, 2.85:1, 2.90:1, 2.95:1, 3:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, and others are considered.

As used herein the term “concentrated hydrogen stream” refers to a hydrogen stream with hydrogen content greater than 90% by mole percent.

As used herein the term “high purity hydrogen stream” refers to a hydrogen stream with hydrogen content greater than 98% by mole percent.

As used herein, the term “co-electrolysis” refers to the process of using electricity to convert water, CO₂ and optionally refinery gas into hydrogen rich syngas or enhanced hydrogen rich syngas, and oxygen.

As used herein, the term “high temperature co-electrolysis” refers to the process of using electricity to convert steam and CO₂ into syngas and oxygen at co-electrolyzer temperatures greater than about 400° C. Temperatures may be between an inclusive range of between about 400° C. and about 1000° C., or any values or subranges therebetween, such as about 600° C. to about 800° C. For example, temperatures (in° C.) of 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, and others are contemplated.

As used herein, the term “refinery gas” refers to vapour streams from one or more unit operations, such as syngas treatment, FT reaction, FT product upgrading, hydrogen removal/separation, etc., which may contain H₂, CO, CO₂, hydrocarbons, and/or inert gases such as nitrogen and argon, and which is re-used in the process.

As used herein, the term “off gas” refers to vapour streams recovered from unit operations, such as hydrogen removal/separation, hydrocarbons upgrading, etc., which may contain H₂, CO, CO₂, hydrocarbons, and/or inert gases such as nitrogen and argon.

As used herein, the term “tail gas” refers to vapour streams recovered from FT unit operations, which may contain H₂, CO, CO₂, hydrocarbons, and/or inert gases such as nitrogen and argon.

As used herein, the term “about” refers to a +/−10% variation from the nominal value. It is to be understood that such a variation is always included in a given value provided herein, whether or not it is specifically referred to.

Carbon-based fossil fuels such as coal, oil and natural gas are non-renewable resources and of limited supply. Combustion of fossil fuel may have contributed to a rise in atmospheric carbon dioxide (CO₂) concentrations, which are believed to contribute to global climate change. The concern for carbon emissions from fossil fuels may have created an increased interest in the development of synthetic fuel sources with low-carbon intensity (CI).

Use of fossil fuels in transport may produce a significant amount of CO₂ emissions and may lead to an increase in its concentration level in the atmosphere. There is a need to reduce CO₂ emissions to minimize its global warming impact. The low CI of synthetic fuels produced from CO₂ from industrial emissions (e.g. cement manufacturing, chemical processes etc.) or even from ambient air may help support efforts to control CO₂ concentration in the atmosphere.

The Fischer-Tropsch (FT) process may convert hydrogen (H₂) and carbon monoxide (CO)—commonly known as syngas-into synthetic hydrocarbons, examples of which include synthetic diesel, naphtha, kerosene, aviation or jet fuel and paraffinic wax. For an effective FT reaction, the molar ratio of the H₂:CO in the syngas may be approximately 2:1.

A solid-oxide electrolytic cell (SOEC) may be capable of electrolytically reducing water and CO₂ into hydrogen, carbon monoxide, and oxygen. Thus, water and CO₂ may be directly converted into syngas. This syngas can then be used as feed to a Fischer-Tropsch reactor. The syngas product stream exiting the SOEC may also comprise unreacted CO₂ and water, as well as by-products such as methane (CH₄) from competing reactions. There may also be a risk of soot formation.

Generally, in the process of co-electrolysis to liquids, hydrogen may be required. One means is the incorporation of a water gas shift (WGS) reaction in the process to generate a stream rich in hydrogen. The feed to the WGS reactor can either be a slipstream of the syngas or a portion of the FT product. The hydrogen may then be separated from the H₂-rich WGS product stream by methods known in the art. However, one of the reaction products of the WGS reaction may be more undesired CO₂.

A separate hydrogen rich syngas streams may also be generated using gas/methane reformers, such as a steam methane reformer (SMR) and/or an autothermal reformer (ATR), and hydrogen may be separated from the resulting product. These processes may mostly rely on fossil fuels.

Another approach is to have a separate water electrolyzer, where the product hydrogen may be used to supply hydrogen to the process. This may require adding an electrolyzer unit to the process.

Accordingly, there is a need for a co-electrolysis-to-liquids (CETL) process with improved carbon efficiency for producing lower CI synthetic hydrocarbons from a suitable syngas source. The process may also produce at least part of the hydrogen required for at least the CETL process itself.

The teachings described herein relate, in at least one embodiment, to a relatively low-carbon intensity process for the production of synthetic hydrocarbons from water, CO₂ and/or or refinery gas, and electricity. A low-carbon intensity fuel may release relatively fewer GHG emissions over its life cycle than the conventional fossil-based fuel that it replaces or is blended with.

The teachings described herein relate to a CETL process for preparing synthetic hydrocarbons, which utilizes relatively low carbon intensity and/or renewable energy to produce oxygen and enhanced hydrogen rich syngas. The oxygen stream may be utilized for preparation of the co-electrolysis feed and the enhanced hydrogen rich syngas is utilized for the production of a hydrogen rich syngas suitable for FT conversions to obtain synthetic hydrocarbons, including transportation fuels and a concentrated hydrogen stream.

The teachings described herein show that utilising the production of an enhanced hydrogen rich syngas by high temperature co-electrolysis to produce a concentrated hydrogen stream and a hydrogen rich syngas and then providing the hydrogen rich syngas to an FT reactor, can provide a process of lower carbon intensity for producing synthetic hydrocarbons.

The teachings described herein show that by operating the solid-oxide electrolytic cell (SOEC) used for the co-electrolysis process step at a H₂O/CO₂ or steam/carbon (S/C) molar ratio that is higher than would conventionally be employed for an FT feed, at least two benefits are realized. Firstly, the enhanced hydrogen rich syngas produced may be used advantageously elsewhere in the CETL process to improve its overall carbon and/or energy efficiency. Secondly, because the higher H₂O:CO₂ molar ratio was found to minimise the production of undesirable CH₄ and soot at moderate temperatures, this may allow the SOEC to be run effectively at moderate temperatures (for example between 600° C. and 800° C.) and hence may extend the lifetime of the SOEC.

For certain embodiments of the CETL process, such as processes 600 and 700, improved outcomes may be observed where the H₂O/CO₂ or steam/carbon (S/C) molar ratio of the inlet feed to the high temperature electrolyzer is greater than 2.0, or more preferably greater than 2.2. The S/C molar ratio may be between an inclusive range of about 2.2 and about 7 or any values or subranges therebetween,, such as between about 3.0 and about 5.0. For example, S/C molar ratios of 2.21:1, 2.22:1, 2.23:1, 2.24:1, 2.25:1, 2.26:1, 2.27:1, 2.28:1, 2.29:1, 2.30:1, 2.35:1, 2.40:1, 2.45:1, 2.50:1, 2.55:1, 2.60:1, 2.65:1, 2.70:1, 2.75:1, 2.80:1, 2.85:1, 2.90:1, 2.95:1, 3:1, 3.05:1, 3.1:1, 3.15:1, 3.2:1, 3.25:1, 3.3:1, 3.35:1, 3.4:1, 3.45:1, 3.5:1, 3.55:1, 3.6:1, 3.65:1, 3.7:1, 3.75:1, 3.8:1, 3.85:1, 3.9:1, 3.95:1, 4.0:1, 4.05:1, 4.1:1, 4.15:1, 4.2:1, 4.25:1, 4.3:1, 4.35:1, 4.4:1, 4.45:1, 4.5:1, 4.55:1, 4.6:1, 4.65:1, 4.7:1, 4.75:1, 4.8:1, 4.85:1, 4.9:1, 4.95:1, 5.0:1, 5.05:1, 5.1:1, 5.15:1, 5.2:1, 5.25:1, 5.3:1, 5.35:1, 5.4:1, 5.45:1, 5.5:1, 5.55:1, 5.6:1, 5.65:1, 5.7:1, 5.75:1, 5.8:1, 5.85:1, 5.9:1, 5.95:1, 6.0:1, 6.05:1, 6.1:1, 6.15:1, 6.2:1, 6.25:1, 6.3:1, 6.35:1, 6.4:1, 6.45:1, 6.5:1, 6.55:1, 6.6:1, 6.65:1, 6.7:1, 6.75:1, 6.8:1, 6.85:1, 6.9:1, 6.95:1, and others are contemplated.

Optionally recycling at least a portion of the water recovered from co-electrolysis or generated in the FT reaction for the co-electrolysis step, and/or recycling excess heat from syngas cooling and/or from the FT reactor to heating the feed to the co-electrolysis step, and/or recycling a refinery gas to generate electricity or heat, or as feed to co-electrolysis step, or a combination thereof may result in an economically viable process with improved carbon efficiency despite seemingly large electrical energy requirements.

The process of the present disclosure need not include the WGS reaction or natural gas reforming, which may help reduce the carbon footprint and dependence on non-renewable feedstocks (e.g. natural gas) as compared to some known processes of producing syngas. Low carbon renewable hydro/solar/wind sourced electricity (which is plentiful and inexpensive in many regions) or low carbon intensity nuclear power can optionally be utilized to reduce and possibly eliminate the need for a non-renewable source, such as natural gas.

The process of the present disclosure involves co-electrolysis of steam and CO₂ in a suitable co-electrolyzer apparatus running at the temperature ranges described herein, which can be referred to as a high-temperature co-electrolyzer, to produce at least an enhanced hydrogen rich syngas. Oxygen may also be produced. The oxygen generated via the co-electrolysis process may be optionally used in an oxyfuel combustion application such as in a power plant operation or calciners and kilns in cement production. The enhanced hydrogen rich syngas generated in the co-electrolysis step is fed to a hydrogen removal step, optionally via any suitable separator apparatus or technique that is operable to separate hydrogen gas from the rest of the flow, and may include one or more of a membrane, pressure swing adsorption (PSA) or absorption operation, to generate a concentrated hydrogen stream and a hydrogen rich syngas.

The hydrogen rich syngas is then reacted in a Fischer Tropsch (FT) reactor to produce an FT product stream that includes at least synthetic hydrocarbons. Optionally water and/or heat may also be produced and/or recovered as part of the FT product stream or as separate by-products.

The CO₂ provided to the co-electrolyzer apparatus can be from any suitable source that can provide process-quality CO₂, such as, for example, an external source such as cement manufacturing, refinery operations, natural gas power production, fermentation processes, direct air capture and/or internal recycle of CO₂ from other portions of the processes described herein.

Optionally, the process water stream generated in the FT reaction may be treated to obtain a level of purity appropriate for subsequent use in other applications or as a recycle stream in the processes described herein. For example, it may then be recycled to a process step, such as the high temperature co-electrolysis step, thereby helping to reduce, and potentially minimizing, the amount of water required from an external source. The recycled water may then be further used as the primary source of water for the co-electrolysis process. The water/steam used in the co-electrolysis process may comprise 50% or more of recycled process water. Recycled process water content of 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36, 37, 38, 39, 40, 41, 42, 43, 44, 45, 46, 47, 48, 49, 50,51, 52, 53, 54, 55, 56, 57, 58, 59, 60, 61, 62, 63, 64, 65, 66, 67, 68, 69, 70, 71, 72, 73, 74, 75, 76,77, 78, 79, 80, 81, 82, 83, 84, 85, 86, 87, 88, 89, 90, 91, 92, 93, 94, 95, 96, 97, 98, 99, or 100% may be used.

A suitable high temperature co-electrolyzer may be selected to conduct the co-electrolysis step as described herein. A suitable operating temperature and/or operating pressure for the co-electrolysis may be selected as appropriate for the type of co-electrolyzer used.

In some embodiments, the co-electrolysis step may be carried out at a temperature from about 400° C. to about 1000° C. Preferably, in some embodiments, the high temperature co-electrolysis step is carried out at temperature above 600° C. to about 800° C.

In some embodiments, the co-electrolysis step can be carried out at a pressure up to 15 bar. In some embodiments, the co-electrolysis step may be carried out at a pressure of up to 35 bar.

In some embodiments, at least two of the CO₂, steam, hydrogen or refinery gas feed streams to the co-electrolyzer may be premixed in a mixer unit.

The hydrogen removal step can be carried out using any suitable process or technique, including, for example a membrane, pressure swing adsorption (PSA) or absorption operation to generate a concentrated hydrogen stream.

The Fischer-Tropsch (FT) reaction is a highly exothermic reaction. At least a portion of energy/heat from the FT reaction, typically in the form of steam, may be used in the process described herein, such as to optionally generate power/electricity.

In some embodiments, the process may comprise feeding at least a portion of the steam generated during the FT reaction to recover heat, which is then used to preheat co-electrolyzer feed streams.

In some embodiments, the process may comprise feeding at least a portion of steam generated in the FT reaction to an electricity generator to produce electricity which can be used to supplement electricity for the co-electrolyzer, and the residual heat after power generation may be used for integration with the process.

In some embodiments, at least a portion of the tail gas produced in the FT reaction may be recycled back to the FT reactor.

Synthetic hydrocarbons obtained from the FT reaction can be subjected to further upgrading processes to obtain desired products. As is known by those skilled in the art, several hydrocarbon treatment methods can form part of the upgrading step depending on the desired refined products, which are essentially free of sulfur. The resulting diesel may be used to produce environmentally friendly, sulfur-free fuel and/or blending stock for fuels by using as is or blending with higher sulfur fuels created from petroleum sources.

In some embodiments, the upgrading process may include hydroprocessing operation(s), such as hydrocracking, thermal cracking, hydrotreating, isomerization or combinations thereof. The hydrocarbons recovered from the upgrading process can be further fractionated to obtain products such as naphtha, diesel, kerosene, jet fuel, lube oil, and wax.

In some embodiments, at least a portion each of the tail gas produced in the FT reaction, off gas produced in the FT product fractionation step, and/or the off gas obtained during hydrogen removal/separation step, or any combinations thereof; may be recycled as a refinery gas for generating electricity for use in co-electrolysis.

In some embodiments, at least a portion of the tail gas produced in the FT reaction, off gas produced in the FT product fractionation step, and/or the off gas obtained during the hydrogen removal/separation step, or any combination thereof may be recycled as a refinery gas to provide additional feed for use in co-electrolysis to generate additional enhanced hydrogen rich syngas.

Co-electrolysis processes may result in generation of heat, which can be recovered. In some embodiments, a portion of the heat generated in the co-electrolysis step may be used for generating power for the co-electrolyzer. In some embodiments, a portion of the heat generated in the co-electrolysis step may be used for generating heat for the co-electrolyzer feed streams.

Waste heat from the co-electrolysis step can be captured through organic Rankine cycle (ORC) and/or Sterling cycle generator technology.

In some embodiments, the hot raw syngas may be fed to a steam-generating heat exchanger to produce steam. In some embodiments, the process may comprise utilizing the steam generated in the heat exchanger to produce electricity to operate the co-electrolyzer, thereby reducing the amount of electricity required from an external source and improving the energy efficiency of the process.

In some embodiments, the refinery gas from the FT reaction (i.e. tail gas) and/or from the fractionation process (i.e. off gas) may be used as a purge gas to fuel an internal combustion engine or micro-turbine to generate power for the co-electrolyzer. The waste heat from the internal combustion engine may be captured via waste heat recovery technology.

In some embodiments, the tail gas obtained from the FT reaction, the off gas obtained from the product fractionation and/or the hydrogen removal/separation may be treated to a carbon dioxide removal operation. In some embodiments, the separated CO₂ may be subjected to compression and dehydration for further utilization or sequestration.

In some embodiments, a portion of the hydrogen generated in the hydrogen removal/separation step may be fed to the hydro-processing operation.

In some embodiments, a portion of the concentrated hydrogen stream may be treated in a further PSA unit to produce high purity hydrogen for use in the FT upgrader or marketed as export hydrogen.

Off gases generated during hydro-processing operation(s) may also be used in power generation.

Examples of suitable FT reactors include fixed bed reactors and slurry-bubble reactors, such as tubular reactors, and multiphase reactors with a stationary catalyst phase.

The term “hydrocracking” used herein refers to the splitting of an organic molecule and adding hydrogen to the resulting molecular fragments to form multiple smaller hydrocarbons (e.g., C₁₀H₂₂+H₂→C₄H₁₀ and skeletal isomers+C₆H₁₄). Since a hydrocracking catalyst may be active in hydro-isomerization, skeletal isomerization can occur during the hydrocracking step. Accordingly, isomers of the smaller hydrocarbons may be formed. Hydrocracking a hydrocarbon stream derived from Fischer-Tropsch synthesis preferably takes place over a hydrocracking catalyst comprising a noble metal or at least one base metal, such as cobalt, platinum, cobalt-molybdenum, cobalt-tungsten, nickel-molybdenum, or nickel-tungsten, at a temperature of from about 550° F. to about 750° F. (from about 288° C. to about 400° C.) and at a hydrogen partial pressure of about 500 psia to about 1,500 psia (about 3,400 kPa to about 10,400 kPa).

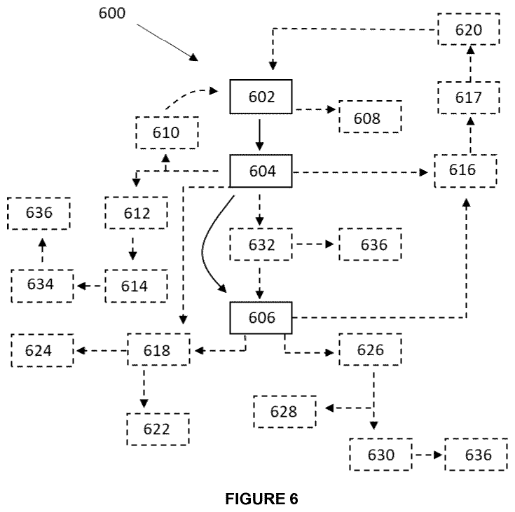

Referring to FIG. 6, one example of a process 600 for preparing synthetic hydrocarbons, that can utilize the systems described herein or other suitable apparatuses and hardware configurations, includes a step 602 of electrolyzing an incoming feed stream of material that can includes a mixture of steam and CO₂ and can be configured/mixed to have a H₂O/CO₂ (S/C) molar ratio greater than about 2.0 in a high-temperature co-electrolyzer apparatus operating at a co-electrolyzer temperature to produce an enhanced hydrogen rich syngas having a first concentration of H₂. The feed stream may, in some examples, include additional components such as H₂, FT refinery gas and others, in addition to the steam and CO₂.

Process 600 also includes a step 604 of removing at least a portion of H₂ from the enhanced hydrogen rich syngas as described herein to provide a hydrogen rich syngas that has a second concentration of H₂ that is lower than the first concentration of H₂. Optionally, step 604 may also include the removal or separation of additional components that may have been present in the enhanced hydrogen rich syngas such as water, steam and others.

Process 600 also has a step 606 of producing at least an FT product stream that includes the desired synthetic hydrocarbons by reacting the hydrogen rich syngas in a suitable Fischer-Tropsch (FT) reactor, such as those described herein. The synthetic hydrocarbons may be subjected to additional processing/treatment steps in some examples of the processes described herein, such as further upgrading and/or product fractionation (not pictured) to modify the concentration and/or composition of the synthetic hydrocarbons.

Process 600 may optionally include one or more recovery steps in which at least some products or outputs from one process step are utilized as an input into another process setup. This may help improve the overall efficiency of the process 600, and/or may reduce waste/byproducts. One example of such a recovery step is illustrated herein as a recovery step 608 for recovering an oxygen stream from the high temperature co-electrolyzer apparatus in step 602.

Optionally, a portion of H₂ removed in step 604 may be removed as a concentrated hydrogen stream. In such arrangements, the process 600 may further include a recycling step 610 for recycling at least a portion of the concentrated hydrogen stream into a different step in the process, such as by feeding the concentrated hydrogen stream into the high temperature co-electrolyzer apparatus of step 602. In some cases, process 600 includes an optional purification step 612 for purifying at least a portion of the concentrated hydrogen stream to help produce a stream with a relatively higher purity of hydrogen than the untreated concentrated hydrogen stream.

Optionally, the process 600 may further include an off gas recovery step 614 for recovering an off gas stream produced by the PSA unit.

In some cases, process 600 has a process water recovery step 616 for recovering a process water stream, process steam or both, produced during steps 602 or 604. Process 600 may have a process water recycling step 617 to recycle one or more process water stream to provide part of the feed stream of step 602. This may help reduce the amount of fresh, make up water that is used in the process. Process 600 may have a process water stream treatment step 620 for treating process water stream by a water treatment process block prior to use in the feed stream in step 602.

Process 600 may have a process steam recycling step 618 for recycling steam for use in generating electricity in step 622, providing heat for the high temperature co-electrolyzer apparatus in step 624, or both. This may help improve the overall efficiency of the process and may reduce the energy that would be otherwise needed to heat the co-electrolyzer apparatus. In step 624 and others, process steam may pass through one or more heat exchangers to provide heat for various steps or apparatuses, such as the high temperature co-electrolyzer apparatus. The electricity generated in step 622 may be used to power apparatuses in one or more steps of the process, for example the high temperature co-electrolyzer apparatus and others. This may help improve the overall efficiency of the system and may help reduce the overall energy consumption of the process.

Process 600 may further include a FT refinery gas recovery step 626 for recovering a FT refinery gas stream from the FT reactor during step 606. In some cases, a recycling step 628 is used to recycle at least a portion of the FT refinery gas to generate electric power for the high temperature co-electrolyzer apparatus, to provide part of the feed stream, or to generate heat energy, or any combination thereof.

One or more CO₂ removal steps may be used to remove CO₂ from one or more streams. A CO₂ removal step 630 may be used to remove at least a portion of CO₂ from the FT refinery gas stream. In some cases, CO₂ removal step 632 is used to remove at least a portion of CO₂ from the hydrogen-rich syngas prior to producing the at least an FT product stream in the FT reactor of step 606. At least a portion of CO₂ from the off gas stream may be removed in a removal step 634. One or more of CO₂ removal steps may have a further CO₂ recycling step 636 to recycle at least part of the CO₂ to provide at least part of the feed stream.

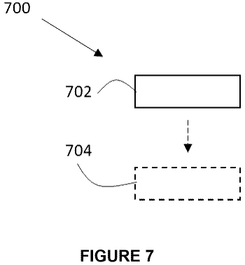

Referring to FIG. 7, process 700 for reducing methane and soot content during production of an enhanced hydrogen rich syngas comprises a step 702 of electrolyzing a feed stream comprising steam and CO₂ with a H₂O/CO₂ (S/C) molar ratio greater than about 2.0 in a high temperature co-electrolyzer apparatus operating at a co-electrolyzer temperature to produce an enhanced hydrogen rich syngas comprising carbon monoxide (CO) and having a first concentration of H₂. The enhanced hydrogen rich syngas in said processes has an H₂:CO molar ratio of greater than 2.2:1. The feed stream may comprise additional components such as H₂, FT refinery gas and others. In some cases, process 700 has a step 704 of removing at least a portion of H₂ from the enhanced hydrogen rich syngas to provide a hydrogen rich syngas having a second concentration of H₂ that is lower than the first concentration of H₂.

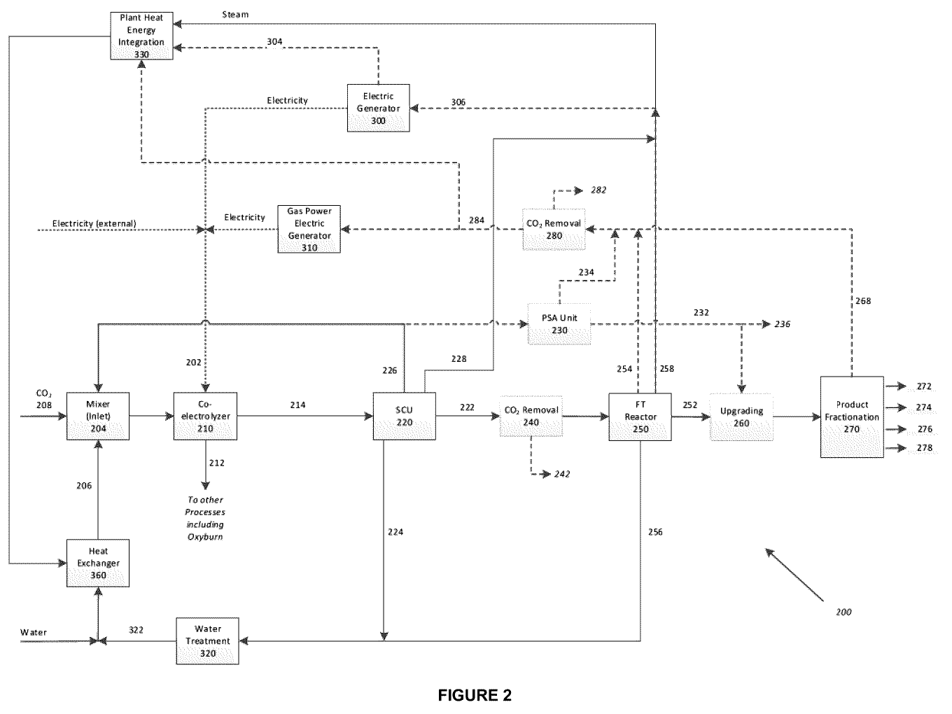

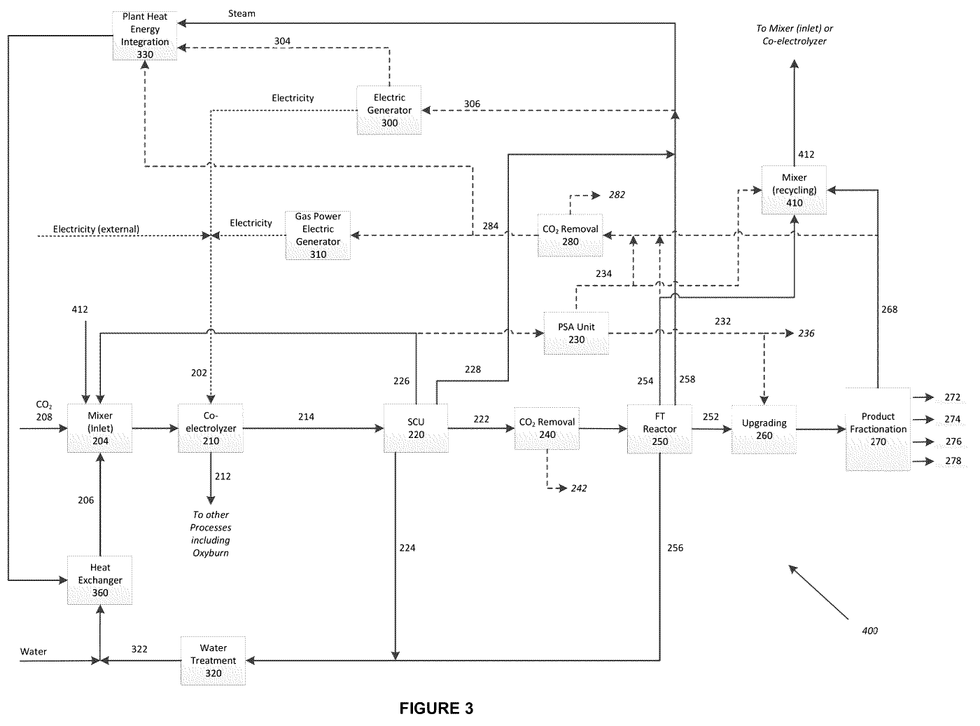

Embodiments of processes 600 and 700 can be implemented using the systems that are schematically depicted in FIG. 2 and FIG. 3 and described herein.

To gain a better understanding of the processes and apparatuses described herein, the following examples are set forth. It will be understood that these examples are intended to describe illustrative embodiments and are not intended to limit the scope of the disclosure in any way.

### EXAMPLES

### Example 1: Prior Art Process

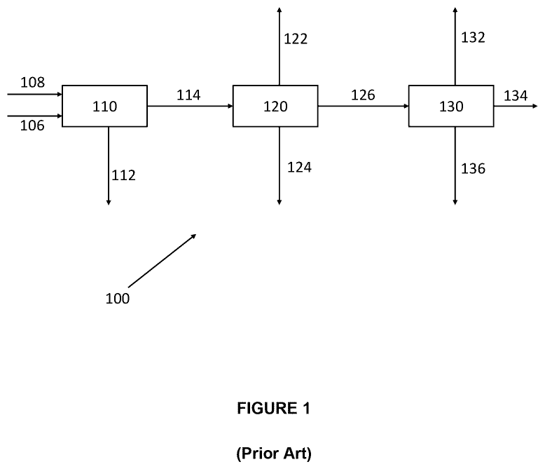

Referring to FIG. 1, which depicts a flow diagram for a prior art process for a co-electrolysis to liquids process. The process is generally denoted by numeral 100 and begins with co-electrolysing a mixture of steam 106 and CO₂ 108 in a high temperature co-electrolyzer 110 to generate oxygen 112 and hydrogen rich syngas 114.

Water 122, and optionally CO₂ 124, is then removed from the hydrogen rich syngas 114 in a separation and/or conditioning unit 120 before the processed hydrogen rich syngas 126 is transferred to a FT reactor 130 to produce the refinery gas 132, hydrocarbon products 134 and water/steam 136. The resulting hydrocarbons 134 are then passed on to a hydrocarbon cracking stage (not shown) to obtain the desired hydrocarbon products, such as naphtha, diesel etc. The diesel formulated in this process is commonly known as synthetic diesel. In addition, an external source of hydrogen can be supplemented to the hydrocarbon cracking unit (not shown).

### Example 2

Referring to FIG. 2, which depicts a flow diagram of an embodiment of a new process, that is generally denoted by numeral 200 and begins with supplying electricity 202 to a high temperature co-electrolyzer apparatus 210 and electrolysing steam 206 and a feed stream of CO₂ 208 in the co-electrolyzer 210 to generate oxygen stream 212 and an enhanced hydrogen rich syngas 214. The CO₂ 208 feed can be additionally sourced from within the CETL process (such as from optional CO₂ removal process blocks 240 and 280). The enhanced hydrogen rich syngas 214 has hydrogen in excess to that which may be required for optimum hydrogen rich syngas 222 as feed for the FT process. This enhancement may be achieved by operating the co-electrolyzer 210 at steam/CO₂ ratios (S/C) of greater than 2.2 or up to 7.0 (or more preferably between 3.0 to 5.0).

The enhanced hydrogen rich syngas 214 is then fed to a separator apparatus, such as a hydrogen separation and conditioning unit 220. The separation and conditioning unit (SCU) 220, which may comprise of a water knockout unit to produce a process water stream 224 and a membrane, PSA unit or absorption unit, is provided to treat the enhanced hydrogen rich stream 214 and produce a hydrogen rich syngas stream 222 and a concentrated hydrogen stream 226. Energy/heat from the hydrogen separation and conditioning unit 220, typically in the form of process steam 228 may be used to heat feed streams to, for example, the co-electrolyzer or FT reactor through plant heat/energy integration 330.

At least a portion of the concentrated hydrogen stream 226 can be recycled back to the co-electrolyzer 210 via the mixer 204, and/or at least a portion of the concentrated hydrogen stream 226 can be optionally treated in a further PSA unit 230 to produce high purity hydrogen 232 for use in the FT upgrader unit 260 or to be marketed as export hydrogen product 236. The off gas stream 234 from the PSA unit 230 can be co-mingled with any of off gas 268 or tail gas 254 or any combination thereof. Concentrated hydrogen stream 226 may be recycled back to the co-electrolyzer 210 to provide a more reducing environment.

Optionally the hydrogen rich syngas stream 222 is passed through a CO₂ removal unit 240 before entering a Fischer-Tropsch reactor 250. The removed CO₂ 242 may be used as a source stream for the co-electrolyzer 210 or for other processes.

The hydrogen rich syngas 222 is then reacted in the Fischer-Tropsch reactor 250 to produce hydrocarbons 252 and process water 256. The hydrocarbons 252 are then subjected to optional upgrading operation(s) in an FT upgrader 260, followed by product fractionation 270 to obtain the desired hydrocarbon products, such as naphtha 272, synthetic diesel 274, synthetic jet 276, and or wax 278.

Process water stream 256 is optionally treated separately or in combination with process water stream 224 in a water treatment process block 320 to form a treated water stream 322. This can be recycled to the co-electrolyzer 210 and be used as a primary water source for co-electrolysis. The water/steam used in the co-electrolysis process may comprise 50% or more of recycled process water. Recycled process water content of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be used.

Optionally, tail gas 254 obtained from the FT reaction, off gas 268 obtained from the product fractionation, off gas 234 from the PSA unit 230 or any combinations thereof can be treated through a CO₂ removal 280.

High purity CO₂, such as 242 and 282, optionally removed by CO₂ units 240 and/or 280, respectively, can be subjected to compression and dehydration for further utilization, such as recycling back to the co-electrolyzer, or for sequestration (not shown).

Energy/heat from the FT reactor 250, typically in the form of process steam 258 from the FT reactor or refinery gas 284 may be used to heat feed streams to, for example, the co-electrolyzer or FT reactor through plant heat/energy integration 330.

Alternatively, process steam 258 from the FT reactor 250 can be directed to the electric generator 300, as shown using optional stream 306, to produce electricity to supplement electricity 202 for the co-electrolyzer 210, and optionally a portion of the residual steam 304 after electricity generation is passed through a heat exchanger, such as heat exchanger 360, or the like in a plant energy heat integration system 330 to recover residual heat for additional use in the process.

Optionally, refinery gas 284 (for example as arising from any of off gas 268, tail gas 254, off gas 234 streams or any combinations thereof) can be used in a power generator, such as an internal combustion engine or micro-turbine in a gas power electric generator unit 310 to generate power for co-electrolyzer 210, or for other purposes. The waste heat from the internal combustion engine can be captured to produce additional electricity using suitable hardware (not shown).

### Example 3

FIG. 3 depicts a schematic flow diagram of further embodiment of the process. The system and process described in relation to this figure is generally denoted by numeral 400 and follows the process flow of FIG. 2 but additionally, at least a portion of the off gas 268, tail gas 254, off gas from hydrogen removal/separation 234 or any combination thereof, can be recycled to the inlet mixer 204 as feed for the co-electrolyzer 210. Optionally, at least a portion off gas 268, tail gas 254, off gas from hydrogen removal/separation 234 or combinations thereof is mixed in the recycle mixer 410 before being fed as refinery gas 412 to the co-electrolyzer.

FIGS. 2 and 3 are schematic representations of embodiments of the process and systems illustrated therein. Parts of the system apparatus have been omitted for clarity, such as pumps, valves, and others. Although the process is shown and described as a generally continuous flow operation, it may alternatively be configured/operated as a batch-type operation with additional storage vessels or valves as needed to achieve a batch-type operation. One or more steps of the process may be repeated, for example a water treatment step.

### Example 4

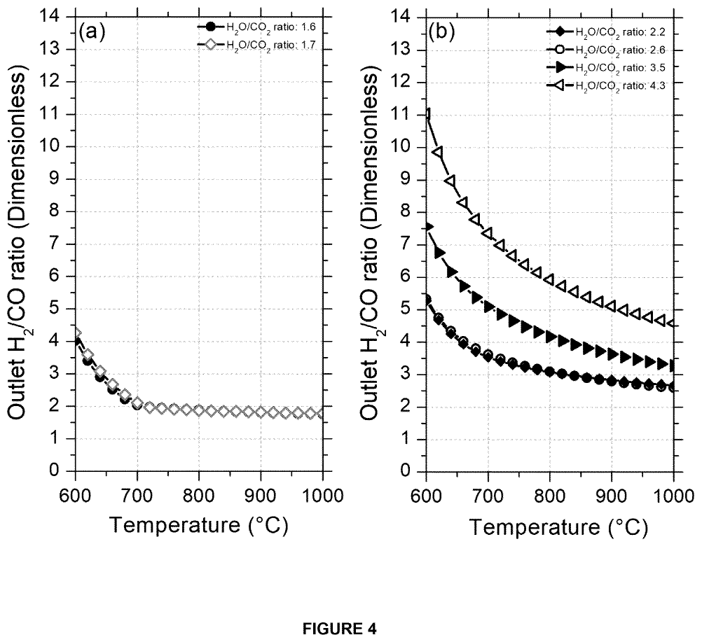

FIG. 4 shows data for the outlet H₂:CO ratio of the high temperature co-electrolyzer syngas product stream as a function of temperature and inlet H₂O/CO₂ (S/C) molar ratio at a pressure of 1 bar and a H₂O/CO₂ decomposition degree of 80%. FIG. 4a) shows the results with an inlet H₂O/CO₂ (S/C) molar ratio of less than 2.1, whilst FIG. 4b) shows the effect of increasing the H₂O/CO₂ (S/C) molar ratios to several values from 2.2 to 4.3. At moderate temperatures (between 600° C. and 800° C.) and an inlet H₂O/CO₂ molar ratio between 2.2 and 4.3, an enhanced hydrogen rich syngas can be produced when compared with that produced using a lower inlet H₂O/CO₂ (S/C) molar ratio.

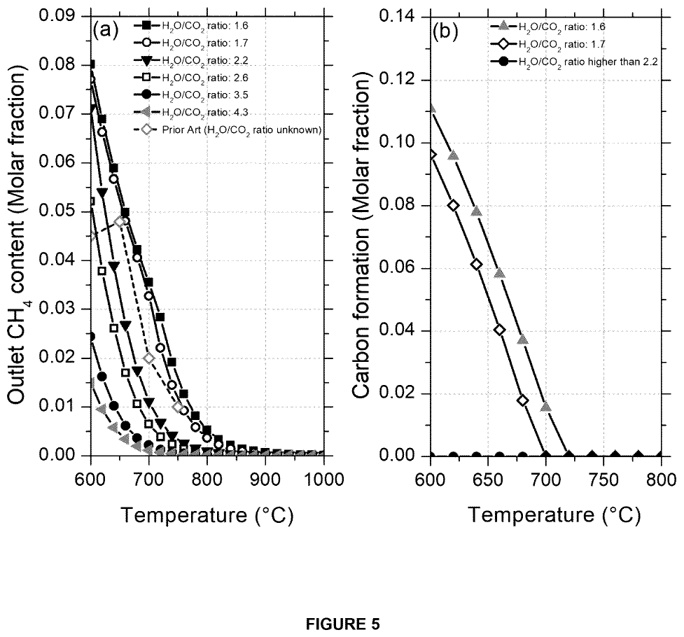

FIG. 5 shows data for methane (CH₄) and carbon formation molar content in the cathode outlet stream of the electrolyser as a function of the inlet feed H₂O/CO₂ (S/C) ratio and temperature. For FIG. 5a), the H₂O/CO₂ (S/C) ratios are values between 1.6 and 4.3; and for FIG. 5b) the H₂O/CO₂ (S/C) ratio is set at 1.6, 1.7 and higher than 2.2. In all cases the pressure was maintained constant at 1 bar. FIG. 5a) shows that the methane content of the outlet stream can be strongly influenced by the inlet H₂O/CO₂ (S/C) molar ratio at moderate temperatures (between 600° C. and 800° C.), with higher S/C values in the range explored showing the most desired effect. FIG. 5b) also shows the positive impact of an inlet H₂O/CO₂ (S/C) molar ratio greater than 2.2 on undesirable carbon formation.

While the teachings herein include illustrative embodiments and examples of some aspects of the disclosure, the description is not intended to be construed in a limiting sense. Thus, various modifications of the illustrative embodiments, as well as other embodiments, may be apparent to persons skilled in the art upon reference to this description.

All publications, patents, and patent applications referred to herein are incorporated by reference in their entirety to the same extent as if each individual publication, patent, or patent application was specifically and individually indicated to be incorporated by reference in its entirety.

1. A process for preparing synthetic hydrocarbons, the process comprising:
a) electrolyzing a feed stream comprising steam and CO₂ with a H₂O/CO₂ (S/C) molar ratio greater than about 2.0 in a high-temperature co-electrolyzer apparatus operating at a co-electrolyzer temperature to produce an enhanced hydrogen rich syngas having a first concentration of H₂;
b) removing at least a portion of H₂ from the enhanced hydrogen rich syngas to provide a hydrogen rich syngas having a second concentration of H₂ that is lower than the first concentration of H₂; and
c) producing at least an FT product stream that comprises synthetic hydrocarbons by reacting the hydrogen rich syngas in a Fischer-Tropsch (FT) reactor.

2. The process of claim 1, wherein the S/C molar ratio is greater than about 2.2.

3. The process of claim 2, wherein the S/C molar ratio is between about 3.0 and about 5.0.

4. The process of any one of claims 1-3, wherein the co-electrolyzer temperature is between about 400° C. and about 1000° C.

5. The process of claim 4, wherein the co-electrolyzer temperature is between about 600° C. and about 800° C.

6. The process of any one of claims 1-5, wherein the enhanced hydrogen rich syngas has an H₂: CO molar ratio of greater than 2.2:1.

7. The process of claim 6, wherein the H₂:CO molar ratio is between about 2.3:1 and about 7:1.

8. The process of any one of claims 1-7, further comprising recovering an oxygen stream from the high temperature co-electrolyzer apparatus in step a).

9. The process of any one of claims 1-8, wherein the portion of H₂ is removed as a concentrated hydrogen stream.

10. The process of claim 9, further comprising recycling at least a portion of the concentrated hydrogen stream into the high temperature co-electrolyzer apparatus.

11. The process of any one of claims 9-10, further comprising purifying at least a portion of the concentrated hydrogen stream to produce high purity hydrogen.

12. The process of claim 11, wherein the at least a portion of the concentrated hydrogen stream is purified via a pressure swing adsorption (PSA) unit.

13. The process of claim 12, further comprising recovering an off gas stream produced by the PSA unit.

14. The process of any one of claims 1-13, wherein the at least a portion of H₂ removed in step b) is removed via a separator apparatus comprising one or more of a membrane, a pressure swing adsorption (PSA) unit or an absorption operation.

15. The process of any one of claims 1-14, further comprising recovering a process water stream, process steam or both, during steps b) or c).

16. The process of claim 15, wherein the process water stream is recycled to provide part of the feed stream of step a).

17. The process of claim 16, wherein the process water stream is treated by a water treatment process block prior to use in the feed stream.

18. The process of any one of claims 16-17, wherein a primary source of the steam in the feed stream is provided by the process water stream.

19. The process of claim 15, wherein the process steam is recycled for use in generating electricity, providing heat for the high temperature co-electrolyzer apparatus, or both.

20. The process of claim 19, wherein the process steam is passed through one or more heat exchangers to provide heat for the high temperature co-electrolyzer apparatus.

21. The process of any one of claims 1-20, further comprising recovering a FT refinery gas stream from the FT reactor during step c).

22. The process of claim 21, further comprising recycling at least a portion of the FT refinery gas to generate electric power for the high temperature co-electrolyzer apparatus, to provide part of the feed stream, or to generate heat energy, or any combination thereof.

23. The process of any one of claims 21-22, further comprising removing at least a portion of CO₂ from the FT refinery gas stream.

24. The process of any one of claims 1-23, further comprising removing at least a portion of CO₂ from the hydrogen-rich syngas prior to producing the at least an FT product stream in the FT reactor.

25. The process of claim 13, further comprising removing at least a portion of CO₂ from the off gas stream.

26. The process of any one of claims 23-25, further comprising recycling the portion of CO₂ to provide part of the feed stream.

27. The process of any one of claims 1-26, wherein the high temperature co-electrolyzer comprises a solid oxide electrolytic cell (SOEC).

28. A process for reducing methane and soot content during production of an enhanced hydrogen rich syngas, the process comprising:
electrolyzing a feed stream comprising steam and CO₂ with a H₂O/CO₂ (S/C) molar ratio greater than about 2.0 in a high temperature co-electrolyzer apparatus operating at a co-electrolyzer temperature to produce an enhanced hydrogen rich syngas comprising carbon monoxide (CO) and having a first concentration of H₂;
wherein the enhanced hydrogen rich syngas has an H₂:CO molar ratio of greater than 2.2:1.

29. The process of claim 28, wherein the S/C molar ratio is greater than about 2.2.

30. The process of claim 29, wherein the S/C molar ratio is between about 3.0 and about 5.0.

31. The process of any one of claims 28-30, wherein the co-electrolyzer temperature is between about 400° C. and about 1000° C.

32. The process of claim 31, wherein the co-electrolyzer temperature is between about 600°° C. and about 800° C.

33. The process of any one of claims 28-32, further comprising removing at least a portion of H₂ from the enhanced hydrogen rich syngas to provide a hydrogen rich syngas having a second concentration of H₂ that is lower than the first concentration of H₂.
